(19) United States
(12) Patent Application Publication
(10) Pub. No.: US 2005/0119752 A1
(43) Pub. Date: Jun. 2, 2005

(54) Artificial intervertebral disc

(75) Inventors: Michael S. Williams, Santa Rosa, CA (US); Jeffrey A. Smith, Santa Rosa, CA (US); Kevin D. Holbrook, Chapel Hill, CA (US)

Correspondence Address: DEANNA J. SHIRLEY, 3418 BALDWIN WAY, SANTA ROSA, CA, 95403, US

(73) Assignee: SyneCor LLC

(21) Appl. No.: 10/990,158

(22) Filed: Nov. 16, 2004

Related U.S. Application Data
(60) Provisional application No. 60/535,954, filed on Jan. 12, 2004.
(60) Provisional application No. 60/523,578, filed on Nov. 19, 2003.

(51) Int. Cl. A61F 2/44

(52) U.S. Cl. 623/17.16; 623/17.11

(57) ABSTRACT

Devices and methods for manufacturing devices for treating degenerated and/or traumatized intervertebral discs are disclosed. Artificial discs and components of discs may include an artificial nucleus and/or an artificial annulus and may be comprised of shape memory materials synthesized to achieve desired mechanical and physical properties. An artificial nucleus and/or annulus according to the invention may comprise one or more hollow bodies that may be filled with a curable material for deployment. A hollow body according to the invention may comprise one or more partitions to define one or more chambers and may comprise means for directing the flow of material within said hollow body.

### RELATED APPLICATIONS

This application is related to and claims the benefit of the priority dates of U.S. Provisional Patent Application Ser. No. 60/535,954 entitled “Artificial Intervertebral Disc”, filed Jan. 12, 2004; and U.S. Provisional Patent Application Ser. No. 60/523,578 entitled “Highly Convertible Endolumenal Prostheses and Methods of Manufacture”, filed Nov. 19, 2003.

### FIELD OF THE INVENTION

The invention herein relates generally to medical devices and methods of treatment, and more particularly to devices and methods used in the treatment of a degenerated intervertebral disc.

### BACKGROUND OF THE INVENTION

Intervertebral disc degeneration is a leading cause of pain and disability, occurring in a substantial majority of people at some point during adulthood. The intervertebral disc, comprising primarily the nucleus pulposus and surrounding annulus fibrosus, constitutes a vital component of the functional spinal unit. The intervertebral disc maintains space between adjacent vertebral bodies, absorbs impact between and cushions the vertebral bodies. The disc allows for fluid movement between the vertebral bodies, both subtle (for example, with each breath inhaled and exhaled) and dramatic (including rotational movement and bending movement in all planes.) Deterioration of the biological and mechanical integrity of an intervertebral disc as a result of disease and/or aging may limit mobility and produce pain, either directly or indirectly as a result of disruption of the functioning of the spine. Estimated health care costs of treating disc degeneration in the United States exceed $60 billion annually.

Age-related disc changes are progressive, and, once significant, increase the risk of related disorders of the spine. The degenerative process alters intradiscal pressures, causing a relative shift of axial load-bearing to the peripheral regions of the endplates and facets of the vertebral bodies. Such a shift promotes abnormal loading of adjacent intervertebral discs and vertebral bodies, altering spinal balance, shifting the axis of rotation of the vertebral bodies, and increasing risk of injury to these units of the spine. Further, the transfer of biomechanical loads appears to be associated with the development of other disorders, including both facet and ligament hypertrophy, osteophyte formation, lyphosis, spondylolisthesis, nerve damage, and pain.

In addition to age-related changes, numerous individuals suffer trauma-induced damage to the spine including the intervertebral discs. Trauma induced damage may include ruptures, tears, prolapse, herniations, and other injuries that cause pain and reduce strength and function.

Non-operative therapeutic options for individuals with neck and back pain include rest, analgesics, physical therapy, heat, and manipulation. These treatments fail in a significant number of patients. Current surgical options for spinal disease include discectomy, discectomy combined with fusion, and fusion alone. Numerous discectomies are performed annually in the United States. The procedure is effective in promptly relieving significant radicular pain, but, in general, the return of pain increases proportionally with the length of time following surgery. In fact, the majority of patients experience significant back pain by ten years following lumbar discectomy.

An attempt to overcome some of the possible reasons for failure of discectomy, fusion has the potential to maintain normal disc space height, to eliminate spine segment instability, and eliminate pain by preventing motion across a destabilized or degenerated spinal segment.

However, although some positive results are possible, spinal fusion may have harmful consequences as well. Fusion involves joining portions of adjacent vertebrae to one another. Because motion is eliminated at the treated level, the biomechanics of adjacent levels are disrupted. Resulting pathological processes such as spinal stenosis, disc degeneration, osteophyte formation, and others may occur at levels adjacent to a fusion, and cause pain in many patients. In addition, depending upon the device or devices and techniques used, surgery may be invasive and require a lengthy recovery period.

Consequently, there is a need in the art to treat degenerative disc disease and/or traumatized intervertebral discs, while eliminating the shortcomings of the prior art. There remains a need in the art to achieve the benefit of removal of a non-functioning intervertebral disc, to replace all or a portion of the disc with a device that will function as a healthy disc, eliminating pain, while preserving motion. There remains a need for an artificial disc or other device that maintains the proper intervertebral spacing, allows for motion, distributes axial load appropriately, and provides stability. In addition, an artificial disc requires secure long-term fixation to bone.

Further, there remains a need for an artificial nucleus that can be implanted within the annulus fibrosus, in order to restore normal disc functioning. Such a nucleus must comprise the characteristic lower durometer than the annulus fibrosus, and the annulus fibrosus must comprise the requisite stiffness as compared with the nucleus. Further, there remains a need for an artificial disc that can withstand typical cyclic stresses and perform throughout the life a patient. An artificial disc that can be implanted using minimally invasive techniques is also needed. And finally, a device that is compatible with current imaging modalities, such as Magnetic Resonance Imaging (MRI) is needed.

### SUMMARY OF THE INVENTION

An endoprosthesis for partial or complete replacement of an intervertebral disc is disclosed comprising one or more shape memory polymers, the shape memory polymers synthesized from a first and second monomer selected to impart predetermined properties on said shape memory polymer. The first and second monomers are combined in a ratio to impart predetermined properties on said shape memory polymer. The first and second monomers are selected for molecular weight, hard and soft segments, transition temperature of said hard and soft segments, and other characteristics. The predetermined properties comprise load bearing capability, compressive resistance, stiffness, crystallinity, tensile strength, mechanical strength, durometer, elasticity, strain recovery rate, strain fixity rate, melting temperature, crystallization temperature, cross-linking density, extent of physical cross-linking, extent of covalent bond cross-linking, extent of formation of interpenetrating networks, and heat of fusion, for example.

The artificial discs disclosed herein substantially replicate the functions of a natural, healthy nucleus pulposus, annulus fibrosis, or both. An artificial disc according to the invention may, for example, comprise a disc-like structure that may have a convex portion, and may have one or more securing rims. An artificial disc disclosed herein may have varying durometers, with, for example, a lower durometer in the nucleus region and a higher durometer in the annular region.

An artificial disc may alternatively comprise a hollow membrane in its delivery configuration and a filled membrane in its deployed configuration. The filling material may in addition be selectively cured to form a more rigid structure. The membrane may, after filling, define an artificial nucleus and/or an artificial annulus, may define a single unitary structure with separate internal chambers, or may define separate portions that may be used separately or together. The internal chambers and/or portions may comprise interbody connections, baffles, partitions, and/or internal seams. An artificial disc or nucleus may comprise a particular durometer selected for its suitability to the particular intervertebral disc undergoing treatment, including the level of the vertebra within the spine.

Methods for making an endoprosthesis disclosed herein comprise the steps selecting a first monomer comprising a first set of characteristics that serves as a first parameter in determining the properties of a shape memory polymer; selecting a second monomer comprising a second set of characteristics that serves as a second parameter in determining the properties of a shape memory polymer; determining a desired ratio of said first monomer to said second monomer; synthesizing a shape memory polymer from said first and said second monomer; manufacturing an endoprosthesis for partial or total replacement of an intervertebral disc from said shape memory polymer; setting a permanent shape for said endoprosthesis; setting a temporary shape for said endoprosthesis.

### BRIEF DESCRIPTION OF THE DRAWINGS

FIG. 1A is a perspective view of an embodiment according to the invention in its deployed configuration.

FIG. 1B is a side view of the embodiment of FIG. 1.

FIG. 2A represents a cross section taken along line A-A of FIG. 1B.

FIG. 2B represents the same cross section of an alternative embodiment of the invention.

FIG. 3 illustrates a cross section of the embodiment of FIGS. 1 and 2 after being placed partially in a delivery configuration.

FIG. 4 is a plan view of the embodiment of FIG. 1 in its delivery configuration.

FIG. 5 is a side view of two vertebrae and a cross section of the embodiment of FIG. 1 deployed therebetween.

FIG. 6 is a side view of a two vertebrae and a side view of a cross section of the embodiment of FIG. 2B in its deployed configuration.

FIG. 7 is a perspective view of an embodiment according to the invention.

FIG. 8A-B is a perspective view of an artificial nucleus according to the invention before and after deployment.

FIG. 9 is a plan view of yet another embodiment according to the invention.

FIG. 10 is a plan view of yet another embodiment according to the invention FIG. 11 is a side view of the embodiment of FIG. 10.

FIG. 12 is a perspective view of the embodiment of FIGS. 10 and 11.

FIG. 13A is a perspective view of yet another alternative embodiment according to the invention.

FIG. 13B is a perspective posterior view of the embodiment of FIG. 20A in situ.

FIG. 14 is a perspective view of an alternative embodiment according to the invention in its delivery configuration mounted upon a delivery mandrel.

FIG. 15A is a side view of an embodiment according to the invention in its deployed configuration in situ.

FIG. 15B is a perspective “cut away” view of the embodiment of FIG. 19, taken along line B-B of FIG. 19.

FIG. 16 is an “exploded” in situ view of an embodiment similar to that illustrated in FIGS. 15A and 15B.

FIG. 17 is a posterior perspective “exploded” in situ view of an alternative embodiment according to the invention.

FIG. 18 is a perspective view of an embodiment according to the invention in its deployed configuration.

FIG. 19A is a plan view cross section of an embodiment according to the invention.

FIGS. 19B-19D illustrate three examples of cross section profiles according to the invention.

FIG. 19E illustrates a plan view cross section of an embodiment according to the invention.

FIG. 19F illustrates an exemplary profile cross section of the embodiment of FIG. 19E.

FIG. 20 is a perspective view of an embodiment according to the invention.

FIG. 21 is an “exploded” in situ view of an embodiment according to the invention.

FIG. 22 is an anterior perspective view of the embodiment of FIG. 19 in situ.

FIG. 23 is a perspective “see-through” view of a membrane configuration of an alternative embodiment according to the invention.

FIG. 24 is a perspective view of an alternative membrane configuration according to the invention.

### DETAILED DESCRIPTION OF THE INVENTION

An endoprosthesis known as an artificial disc and/or an artificial disc nucleus are designed to replace a degenerated intervertebral disc. Such an artificial disc or disc nucleus may be expandable and/or self-expanding.

An “expandable” endoprosthesis comprises a reduced profile configuration and an expanded profile configuration. An expandable endoprosthesis according to the invention may undergo a transition from a reduced configuration to an expanded profile configuration via any suitable means, or may be self-expanding. Some embodiments according to the invention may comprise a substantially hollow interior that may be filled with a suitable medium, examples of which are set forth below. Such embodiments may accordingly be introduced into the body in a collapsed configuration, and, following introduction, may be filled to form a deployed configuration. Embodiments according to the invention may accordingly be implanted percutaneously or surgically. If implanted surgically, embodiments according to the invention may be implanted from either an anterior or a posterior approach, following the removal of some or all of the native disc, excepting the periphery of the native nucleus.

“Spinal fusion” is a process by which one or more adjacent vertebral bodies are adjoined to one another in order to eliminate motion across an unstable or degenerated spinal segment.

“Preservation of mobility” refers to the desired maintenance of normal motion between separate spinal segments.

“Spinal unit” refers to a set of the vital functional parts of the spine including a vertebral body, endplates, facets, and intervertebral disc.

The term “cable” refers to any generally elongate member fabricated from any suitable material, whether polymeric, metal or metal alloy, natural or synthetic.

The term “fiber” refers to any generally elongate member fabricated from any suitable material, whether polymeric, metal or metal alloy, natural or synthetic.

As used herein, the term “braid” refers to any braid or mesh or similar wound or woven structure produced from between 1 and several hundred longitudinal and/or transverse elongate elements wound, woven, braided, knitted, helically wound, or intertwined by any manner, at angles between 0 and 180 degrees and usually between 45 and 105 degrees, depending upon the overall geometry and dimensions desired.

Unless specified, suitable means of attachment may include by thermal melt, chemical bond, adhesive, sintering, welding, or any means known in the art.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, radiation, including but not limited to radiation in the ultraviolet range, and others. Some embodiments according to the invention may comprise one or more polymers having a structure that assumes a first configuration, a second configuration, and a hydrophilic polymer of sufficient rigidity coated upon at least a portion of the structure when the device is in the second configuration. Upon placement of the device in an aqueous environment and consequent hydration of the hydrophilic polymer, the polymer structure reverts to the first configuration.

Some embodiments according to the invention, while not technically comprising shape memory characteristics, may nonetheless readily convert from a constrained configuration to a deployed configuration upon removal of constraints, as a result of a material's elasticity, super-elasticity, a particular method of “rolling down” and constraining the device for delivery, or a combination of the foregoing. Such embodiments may comprise one or more elastomeric or rubber materials.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions.

“Transition temperature” refers to the temperature above which a shape memory polymer reverts to its original memorized configuration.

The term “strain fixity rate” R_{f} is a quantification of the fixability of a shape memory polymer's temporary form, and is determined using both strain and thermal programs. The strain fixity rate is determined by gathering data from heating a sample above its melting point, expanding the sample to 200/o of its temporary size, cooling it in the expanded state, and drawing back the extension to 0%, and employing the mathematical formula:
R_{f}(N)=εᵤ(N)/εₘ
where εᵤ(N) is the extension in the tension-free state while drawing back the extension, and εₘ is 200%.
The “strain recovery rate” Rᵣ describes the extent to which the permanent shape is recovered:

$${R}_{r} ( N ) = \frac{{ɛ}_{m} - {ɛ}_{p} ( N )}{{ɛ}_{m} - {ɛ}_{p} ( N - 1 )}$$

where εₚ is the extenstion at the tension free state.

A “switching segment” comprises a transition temperature and is responsible for the shape memory polymer's ability to fix a temporary shape.

A “thermoplastic elastomer” is a shape memory polymer comprising crosslinks that are predominantly physical crosslinks.

A “thermoset” is a shape memory polymer comprising a large number of crosslinks that are covalent bonds.

Shape memory polymers are highly versatile, and many of the advantageous properties listed above are readily controlled and modified through a variety of techniques. Several macroscopic properties such as transition temperature and mechanical properties can be varied in a wide range by only small changes in their chemical structure and composition. More specific examples are set forth in Provisional U.S. Patent Application Ser. No. 60/523,578 and are incorporated in their entirety as if fully set forth herein.

Shape memory polymers are characterized by two features, triggering segments having a thermal transition Tₜᵣₐₙₛ within the temperature range of interest, and crosslinks determining the permanent shape. Depending on the kind of crosslinks (physical versus covalent bonds), shape memory polymers can be thermoplastic elastomers or thermosets. By manipulating the types of crosslinks, the transition temperature, and other characteristics, shape memory polymers can be tailored for specific clinical applications.

More specifically, according the invention herein, one can the control shape memory behavior and mechanical properties of a shape memory polymer through selection of segments chosen for their transition temperature, and mechanical properties can be influenced by the content of respective segments. The extent of crosslinking can be controlled depending on the type of material desired through selection of materials where greater crosslinking makes for a tougher material than a polymer network. In addition, the molecular weight of a macromonomeric crosslinker is one parameter on the molecular level to adjust crystallinity and mechanical properties of the polymer networks. An additional monomer may be introduced to represent a second parameter.

Further, the annealing process (comprising heating of the materials according to chosen parameters including but not limited to time and temperature) increases polymer chain crystallization, thereby increasing the strength of the material. Consequently, according to the invention, the desired material properties can be achieved by using the appropriate ratio of materials and by annealing the materials.

Additionally, the properties of polymers can be enhanced and differentiated by controlling the degree to which the material crystallizes through strain-induced crystallization. Means for imparting strain-induced crystallization are enhanced during deployment of an endoprosthesis according to the invention. Upon expansion of an endoprosthesis according to the invention, focal regions of plastic deformation undergo strain-induced crystallization, further enhancing the desired mechanical properties of the device, such as further increasing radial strength. The strength is optimized when the endoprosthesis is induced to bend preferentially at desired points.

Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)s, especially poly(.beta.-hydroxybutyrate), poly(3-hydroxyoctanoate) and poly(3-hydroxyfatty acids).

Suitable synthetic polymer blocks include polyphosphazenes, poly(vinyl alcohols), polyamides, polyester amides, poly(amino acid)s, synthetic poly(amino acids), polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyesters, polyethylene terephthalate, polysiloxanes, polyurethanes, fluoropolymers (including but not limited to polyfluorotetraethylene), and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

For those embodiments comprising a shape memory polymer, the degree of crystallinity of the polymer or polymeric block(s) is between 3 and 80%, more often between 3 and 65%. The tensile modulus of the polymers below the transition temperature is typically between 50 MPa and 2 GPa (gigapascals), whereas the tensile modulus of the polymers above the transition temperature is typically between 1 and 500 MPa. Most often, the ratio of elastic modulus above and below the transition temperature is 20 or more.

The melting point and glass transition temperature of the hard segment are generally at least 10 degrees C., and preferably 20 degrees C., higher than the transition temperature of the soft segment. The transition temperature of the hard segment is preferably between −60 and 270 degrees C., and more often between 30 and 150 degrees C. The ratio by weight of the hard segment to soft segments is between about 5:95 and 95:5, and most often between 20:80 and 80:20. The shape memory polymers contain at least one physical crosslink (physical interaction of the hard segment) or contain covalent crosslinks instead of a hard segment. The shape memory polymers can also be interpenetrating networks or semi-interpenetrating networks. A typical shape memory polymer is a block copolymer.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

Examples of highly elastic materials including but not limited to vulcanized rubber, polyurethanes, thermoplastic elastomers, and others may be used according to the invention.

Curable materials include any material capable of being able to transform from a fluent or soft material to a harder material, by cross-linking, polymerization, or other suitable process. Materials may be cured over time, thermally, chemically, or by exposure to radiation. For those materials that are cured by exposure to radiation, many types of radiation may be used, depending upon the material. Wavelengths in the spectral range of about 100-1300 nm may be used. The material should absorb light within a wavelength range that is not readily absorbed by tissue, blood elements, physiological fluids, or water. Ultraviolet radiation having a wavelength ranging from about 100-400 nm may be used, as well as visible, infrared and thermal radiation. The following materials are some examples of curable materials: urethanes, polyurethane oligomer mixtures, acrylate monomers, aliphatic urethane acrylate oligomers, acrylamides, UV curable epoxies, photopolymerizable polyanhydrides and other UV curable monomers. Alternatively, the curable material can be a material capable of being chemically cured, such as silicone based compounds which undergo room temperature vulcanization.

Though not limited thereto, some embodiments according to the invention comprise one or more therapeutic substances that will elute from the surface. Suitable therapeutics include but are not limited to bone growth accelerators, bone growth inducing factors, osteoinductive agents, immunosuppressive agents, steroids, anti-inflammatory agents, pain management agents (e.g, analgesics), tissue proliferative agents to enhance regrowth and/or strengthening of native disc materials, and others. According to the invention, such surface treatment and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”).

The use of polymeric materials in the fabrication of endoprostheses confers the advantages of improved flexibility, compliance and conformability. Fabrication of an endoprosthesis according to the invention allows for the use of different materials in different regions of the prosthesis to achieve different physical properties as desired for a selected region. An endoprosthesis comprising polymeric materials has the additional advantage of compatibility with magnetic resonance imaging, potentially a long-term clinical benefit.

As set forth above, some embodiments according to the invention may comprise components that have a substantially hollow interior that may be filled after being delivered to a treatment site with a suitable material in order to place the device in a deployed configuration. Accordingly, such embodiments may comprise a fluid retention bag having a membrane layer comprising polyvinyl chloride (PVC), polyurethane, and or laminates of polyethylene terephthalate (PET) or nylon fibers or films within layers of PVC, polyurethane or other suitable material. Such a fluid retention bag or membrane layer alternatively may comprise Kevlar, polyimide, a suitable metal, or other suitable material within layers of PVC, polyurethane or other suitable material. Such laminates may be of solid core, braided, woven, wound, or other fiber mesh structure, and provide stability, strength, and a controlled degree of compliance. Such a laminate membrane layer may be manufactured using radiofrequency or ultrasonic welding, adhesives including ultraviolet curable adhesives, or thermal energy.

A fluid retention bag as set forth above may be filled with any suitable material including but not limited to saline, contrast media, hydogels, a polymeric foam, or any combination thereof. A polymeric foam may comprise a polyurethane intermediate comprising polymeric diisocyanate, polyols, and a hydrocarbon, or a carbon dioxide gas mixture. Such a foam may be loaded with any of numerous solid or liquid materials known in the art that confer radiopacity.

Such a fluid retention membrane and/or bag may be designed to replace an entire intervertebral disc. Alternatively, it may replace only the nucleus pulposus or only the annulus fibrosus. Such a device may comprise one or more filling ports, and include separate filling ports for the nucleus pulposus and annulus fibrosus, to allow for varying durometers, and possibly varied materials in order to mimic the properties of the native disc components. Further, such a device may comprise a characteristic durometer selected for suitability to the level of the vertebra within the spine for which the intervertebral disc is being treated. For example, an artificial intervertebral disc nucleus within the cervical spine may comprise a lower durometer than a replacement nucleus in the lumbar region.

Such a device may comprise a single unit, or may be two or more individual parts. If the device comprises two or more component parts, the parts may fit together in a puzzle-like fashion. The device may further comprise alignment tabs for stable alignment between the vertebral bodies.

Such a fluid retention membrane and/or bag may comprise interbody connections and/or baffles and/or partitions or generally vertically oriented membranes in order to maintain structural integrity after filling, to increase the devices ability to withstand compressive, shear, and other loading forces, and/or to direct filling material flow and positioning, and/or to partition portions of the disc in order to separate injection of different types or amounts of filling materials.

Following surgical or minimally invasive surgical access and removal of all or a portion of the native disc, a deflated fluid retention bag or membrane may be delivered to the intervertebral space surgically or through a catheter and/or cannula. The membrane and/or bag is positioned within the intervertebral space. The membrane inflation port or ports are then attached to the injection source. Filling material is then injected. Following injection of the filling material, which may be curable by any suitable means or may be catalytically activated or may remain in fluid form, the injection source is detached and removed.

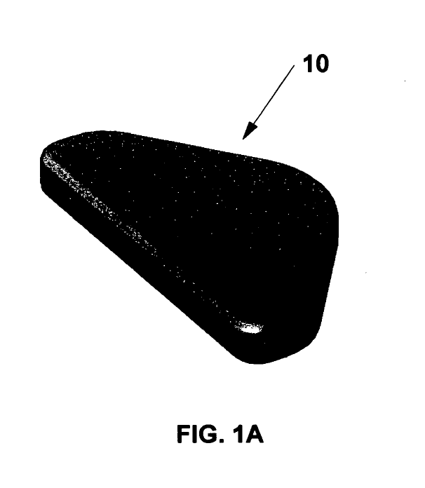

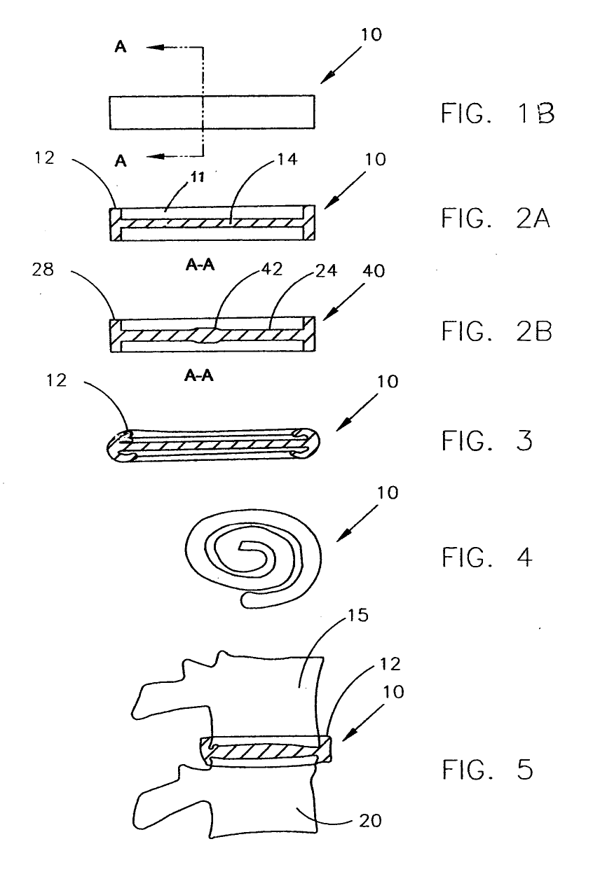

Details of the invention can be better understood from the following descriptions of specific embodiments according to the invention. FIG. 1A illustrates a perspective view of artificial disc 10 according to the invention in its deployed configuration. FIG. 1B illustrates a side view of artificial disc 10 according to the invention in its deployed configuration. In its deployed configuration, cross sectional area of artificial disc 10 is most often between 800 mm and 2000 mm², and between 5.0 mm and 15.0 mm high depending upon the dimensions required of a particular clinical application. A cross section of artificial disc 10 taken along line A-A is illustrated in FIG. 2A. Artificial disc 10 comprises annular rim 12, annular region 11 and nucleus region 14. Nucleus region 14 may comprise properties that differ significantly from annular region 11. More specifically, nuclear region 14 may comprise a lower durometer, more compliant material, corresponding to the properties of a natural nucleus pulposus. In contrast, annular region 11 may comprise a tougher, stiffer, less compliant material with a higher durometer, in order to achieve the objectives of a natural annulus. Overall, the resulting device must be able to withstand loads of between 150N, consistent with a typical load at supine rest, to between 4000N and greater than 6000N, consistent with typical loads experienced during lifting and jumping.

A cross section of an alternative embodiment taken along the same line is shown in FIG. 2B. Artificial disc 40 similarly comprises annular rim 28 and nucleus region 24. However, nucleus region 24 also comprises convex portion 42 disposed generally about a center point of nucleus region 24.

Returning to the embodiment of FIGS. 1 and 2A, artificial disc 10 is illustrated in FIG. 3 following a step of placing artificial disc 10 in its delivery configuration. As shown in cross section in FIG. 3, annular rim 12 is folded down in a step in order to achieve a delivery configuration. Next, artificial disc 10 is “rolled” in order to form an even more compact configuration for delivery, as illustrated in FIG. 4. Alternatively, or in addition, artificial disc 10 may be folded in order to achieve a compact delivery configuration.

In its delivery configuration, artificial disc 10 is most often between 30.0 mm and 70.0 mm in length, 5.0 mm and 25.0 mm wide, and between 5.0 mm and 25.0 mm high, again depending upon the dimensions required of a particular clinical application. Artificial disc 10 may be manufactured from shape memory materials exhibiting properties selectively imparted into the materials, and may transition between its delivery configuration and deployed configuration following change in temperature, hydration, salinity, or the application of heat, radiation, or other initiator.

FIG. 5 depicts the embodiment of FIGS. 1A, 2A, 3 and 4 within a typical treatment site following a partial or complete discectomy. Accordingly, artificial disc 10 is shown in cross section its deployed configuration placed between vertebral bodies 15 and 20. Annular rim 12 secures artificial disc 10 against displacement by surrounding and engaging vertebral bodies 15 and 20, while central region 14 serves to restore and maintain a healthy intervertebral space, absorb axial load, serve as a cushion between vertebral bodies 15 and 20, and otherwise serve the functions much of a healthy intervertebral disc.

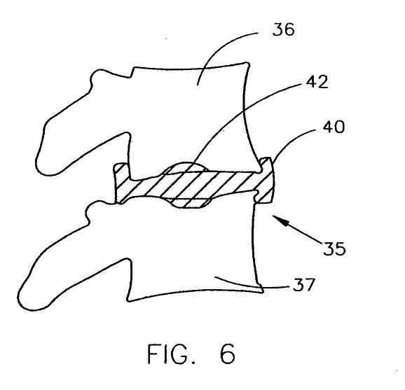

FIG. 6 sets forth another embodiment according to the invention. Artificial disc 35, comprising securing rim 40, and convex portion 42 is shown in its deployed configuration in cross section, situated between vertebral bodies 36 and 37. Convex portion 42 serves to restore the normal intervertebral space and to serve as a shock absorber while allowing a normal range of motion in all planes, including +/−10 degrees flexion, +/−5 degrees extension/lateral bending, and +/−2 degrees rotation. Convex portion 42 further acts as an alignment and nucleus load bearing structure. Convex portion 42 most often comprises materials having a hardness in the range of 20-70 Shore A durometer, most often around 35 Shore A durometer, consistent with the function of convex portion 42 as a substitute nucleus. In contrast, securing rim 40 and the exterior of artificial disc 35 most often comprises materials of a higher durometer of between 35 and 90 Shore A, consistent with the function of these portions as a replacement for the natural annulus fibrosus. Alternatively, the durometer of artificial disc 35 may be varied throughout the device, with a lowest durometer at or near the most central interior portion of the device, with durometer gradually increasing from such point to a highest durometer at the outer annular portions of artificial disc 35.

Such varying durometer may be achieved, for example, according to a process whereby the outer annular region of the artificial disc, comprising one or more curable materials, is cured following delivery of the device. Such curing serves to modify the chemical structure of the material which toughens the portion of the artificial disc simulating the annulus region, thereby increasing the wear properties and increasing the materials' torsional stiffness and/or torsional moment. Such characteristics can alternatively be instilled via either a cross-linking or a catalytically activated process prior to delivery.

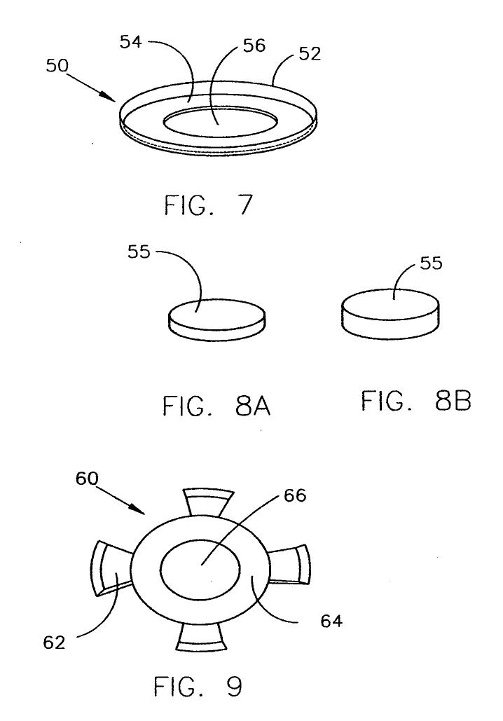

An alternative embodiment according to the invention is illustrated in a perspective view in FIG. 7. Artificial disc 50 comprises annular rim 52 and central region 54. Artificial disc 50 also comprises central void 56. Artificial nucleus 55, illustrated in its delivery configuration in FIG. 8A and in its deployed configuration in FIG. 8B, is designed for either insertion into central void 56 in a second step, or as a stand-alone implant within a native disc annulus where a new nucleus only is required. Artificial disc 50 can thereby accommodate a more compact delivery configuration to facilitate a minimally invasive procedure.

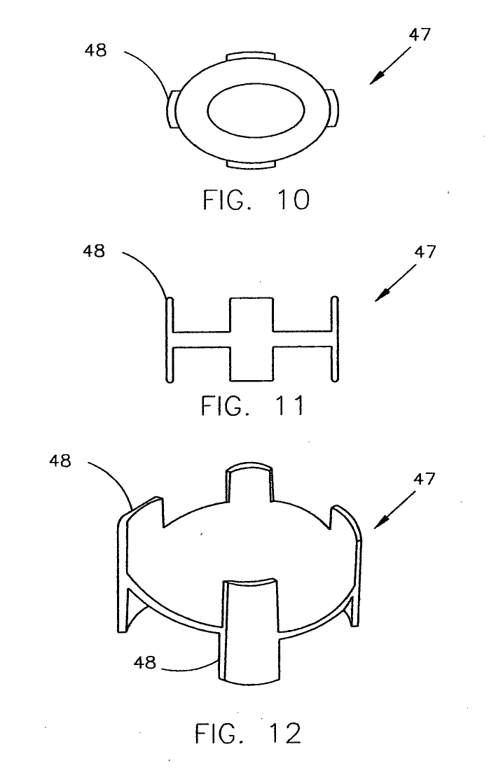

Artificial disc 60 of FIG. 9 similarly comprises central void 66 within central region 64, in which artificial nucleus 55 of FIGS. 8A-8B can be inserted. Artificial disc 60 further comprises engaging tabs 62 for securing artificial disc 60 to a vertebral body (not pictured).

Yet another alternative embodiment is shown in a plan view in FIG. 10, in a side view in FIG. 11, and a perspective view in FIG. 12. Artificial disc 47 comprises securing tabs 48. Securing tabs 48 surround and engage a superior and an inferior vertebral body (not pictured) and affix artificial disc 47 thereto. The disc remains free-floating and the edge tabs keep the device in place by preventing lateral movement of the disc in relation to the superior and inferior vertebral bodies.

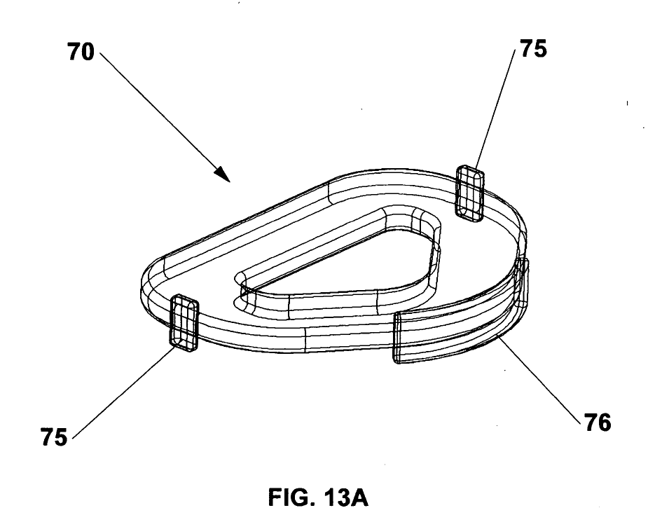

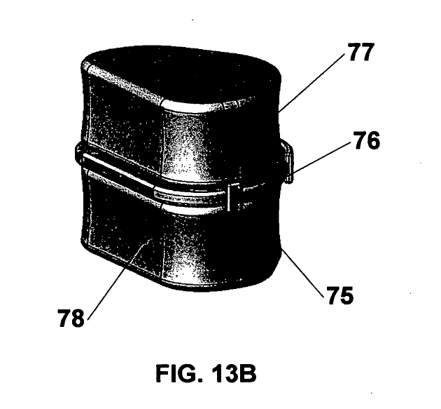

FIGS. 13A and 13B are three-dimensional illustrations of an embodiment similar to that illustrated in FIGS. 10-12. Artificial disc 70, which comprises alignment tabs 75 and anterior alignment tab 76, for the secure alignment of artificial disc 70 within the intervertebral space. Once artificial disc 70 is deployed within the intervertebral space, alignment tabs 75 and anterior alignment tab 76 bear against the superior and inferior vertebral bodies 77 and 78, as illustrated in FIG. 13B.

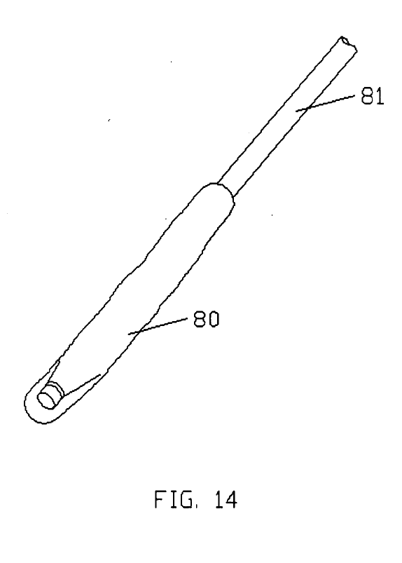

FIGS. 14-24 introduce alternative disc replacement devices according to the invention. FIG. 14 illustrates a perspective view of artificial annulus 80 in its collapsed, unfilled delivery configuration. Artificial annulus 80 generally comprises a fillable membrane that may alternatively be designed to replace both the nucleus pulposus and annulus fibrosus, or the nucleus pulposus alone, as illustrated below.

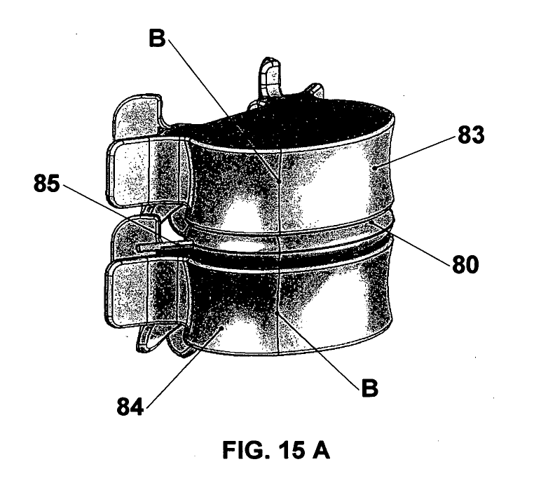

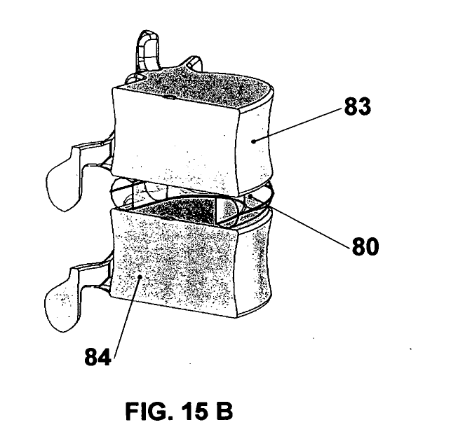

In the delivery configuration, artificial annulus 80 may be delivered to the intervertebral space in any of the suitable methods set forth above. Following delivery to the treatment site, artificial annulus 80 may be filled with a suitable material in order to achieve its deployed configuration, as illustrated in FIG. 15A. Artificial annulus 80, comprising fill port 85 is positioned between vertebral bodies 83 and 84. A liquid or dry polymer may be introduced into the interior of artificial annulus 80 via fill port 85. Following delivery, the polymer will undergo a reaction to change into a solid porous body or gel. Arigid polyurethane foam, for example, will then be in place within the interior of the membrane of artificial annulus 80.

In FIG. 15B, a “cut away” taken along line B-B of FIG. 15A, is shown to better illustrate the position and structure of artificial annulus 80 in situ. Also revealed in FIG. 15B, artificial annulus 80 can be utilized alone or in conjunction with a separate artificial nucleus (not pictured).

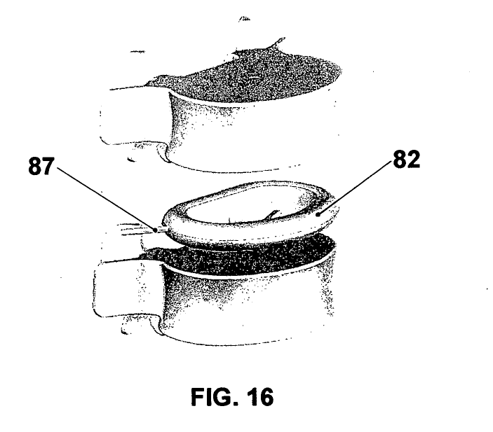

For further illustration of such an embodiment, a three-dimensional “exploded” view of an artificial annulus 82 with fill port 87 is illustrated in FIG. 16.

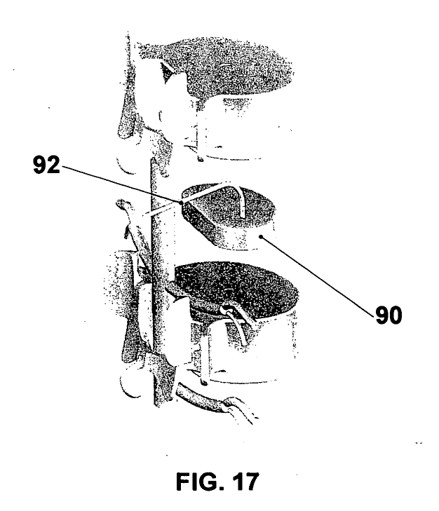

Turning now to FIG. 17, artificial nucleus 90 is illustrated in an exploded view in situ in FIG. 17. As set forth above, an embodiment according to the invention may comprise a nucleus only replacement. Suitable filling material may be introduced into the interior of artificial nucleus 90 via filling port 92. Suitable filling material may comprise liquid or dry polymer that changes into a solid porous structure or gel following introduction. For an artificial nucleus, a lower modulus foam or hydrogel may be most suitable. Accordingly, artificial nucleus 90 will more closely mimic the mechanical properties of a healthy native nucleus pulposus.

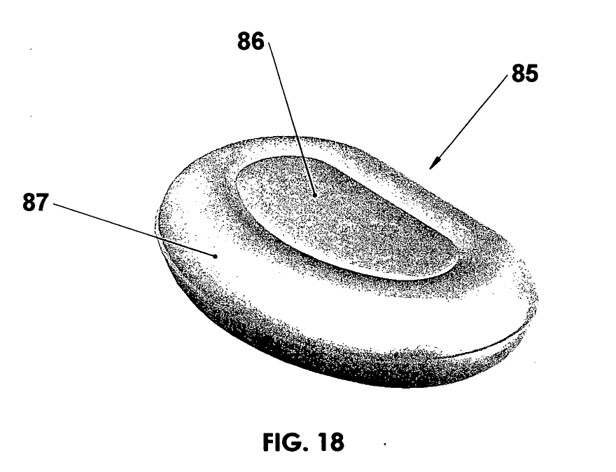

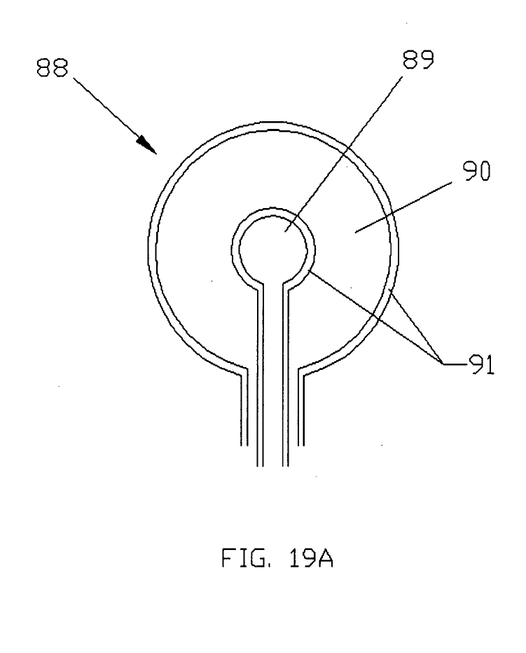

FIG. 18 illustrates an opaque three dimensional perspective view of an embodiment according comprising both of the foregoing components discussed. Artificial disc 85 comprises artificial nucleus 86 and artificial annulus fibrosus 87. Artificial disc 85 may be constructed whereby artificial nucleus 86 and artificial annulus 87 are integral with one another, or, alternatively, as two separate pieces that fit together.

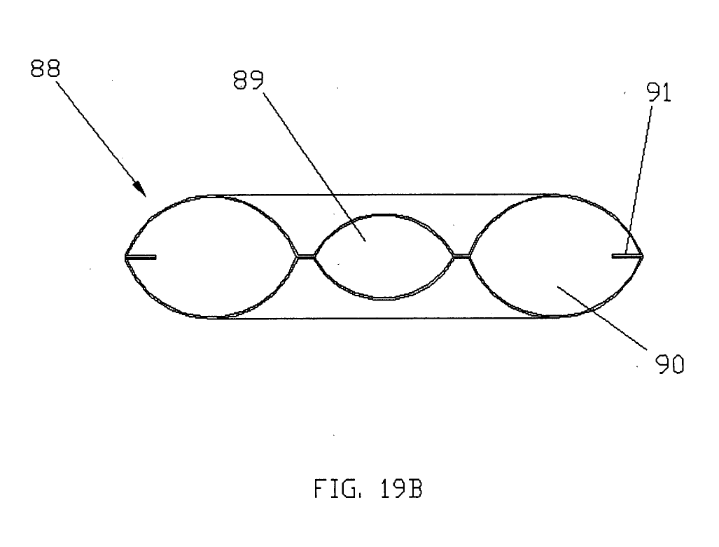

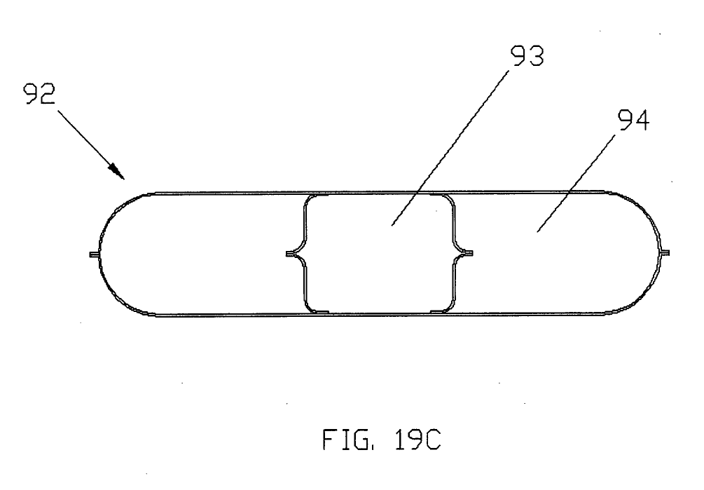

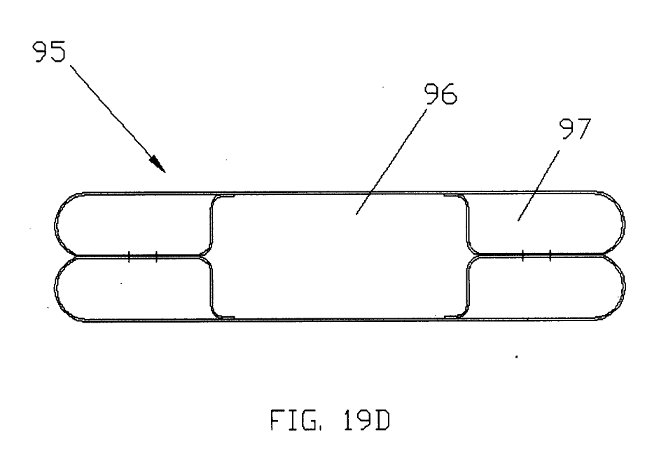

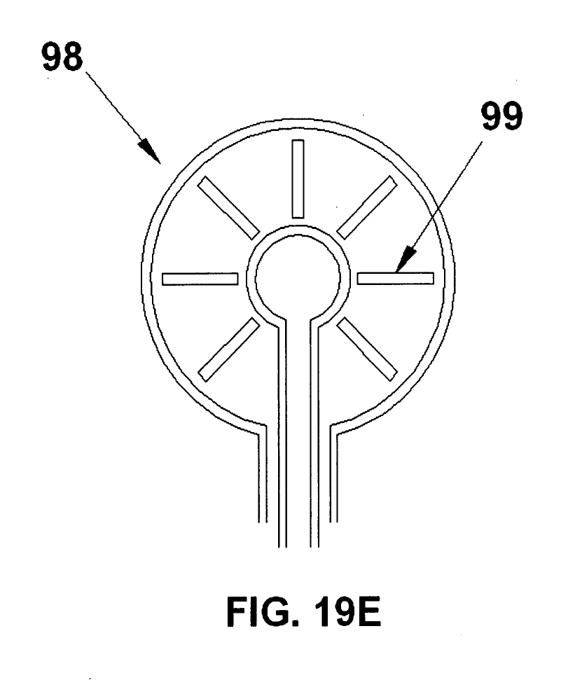

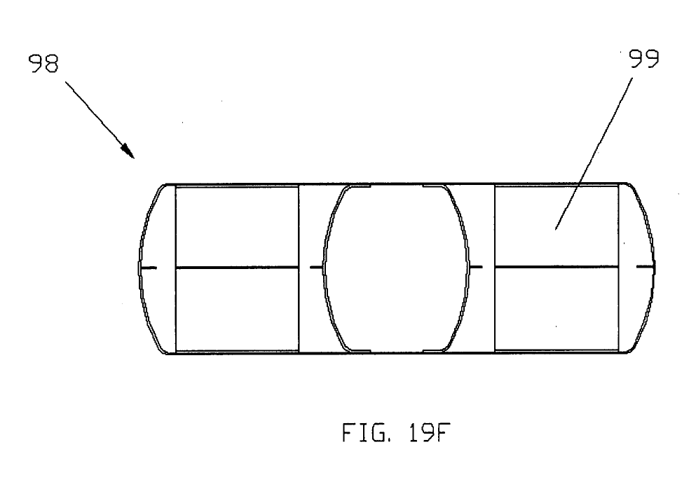

For example, an artificial disc according to the invention may be comprise of a unitary membrane having internal channels leading to separate internal chambers. Examples of the configuration of the internal channels and internal chambers are set forth in FIGS. 19A-19F. Separate internal channels allow the introduction of varying materials into the separate chambers of the member in order to confer varying mechanical properties upon the respective portions of the device. Further, a membrane according to the invention may comprise inverted seams to reduce trauma to body tissues. And as illustrated in FIGS. 19E-19F, an embodiment according to the invention may further comprise baffles to direct fluid flow and to impart stability upon the device.

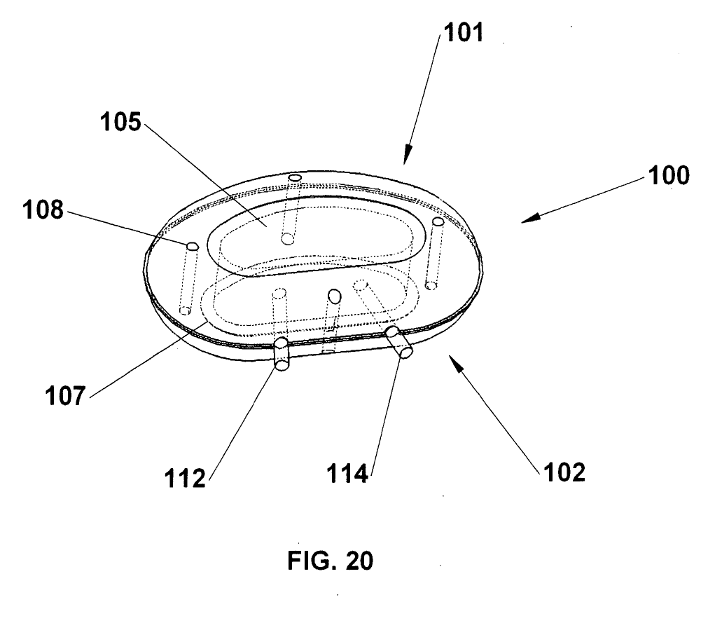

Turning now to FIG. 20, artificial disc 100, comprising component artificial nucleus 105 and artificial annulus 107. Artificial annulus may further comprise superior component 101 and inferior component 102, and internal interbody membrane connections 108 that serve to secure superior component 101 to inferior component 102, and vice versa. Further, nucleus 105 may comprise nucleus filling port 114, and artificial annulus 107 may comprise annulus filling port 112. Separate port for the annulus and the nucleus enable the separate filling of these components. Accordingly, artificial nucleus 105 may be filled with a material that has a lower durometer than a material used to fill artificial annulus 107, whereby artificial nucleus 105 and artificial annulus 107 will more closely replicate the physical and mechanical properties of a healthy native nucleus and annulus respectively.

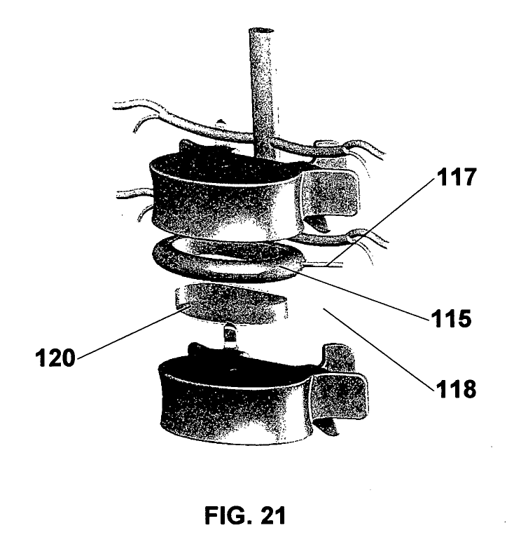

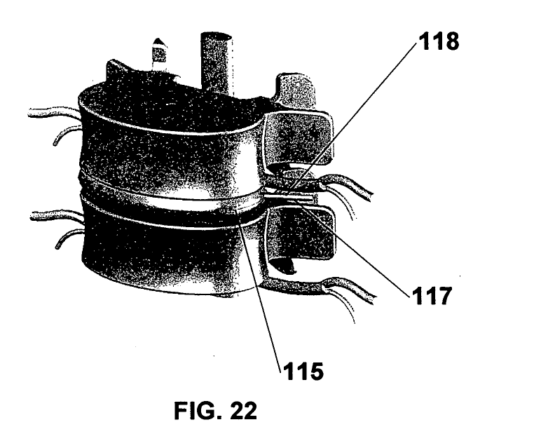

FIG. 21 illustrates via an “exploded” view that separate component artificial annulus 115 and artificial nucleus 120, and illustrates the “mating” of the respective components in situ. Similar to the embodiments set forth above, artificial annulus comprises annulus port 117, and artificial nucleus comprises nucleus port 118. In their delivery configuration, the combined device appears as illustrated in FIG. 22, with artificial annulus 115 encircling the now hidden artificial nucleus.

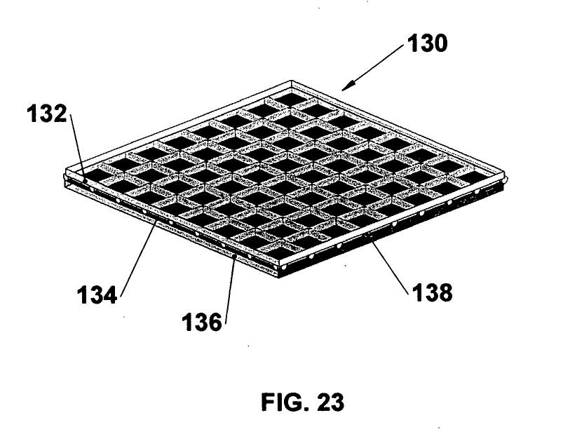

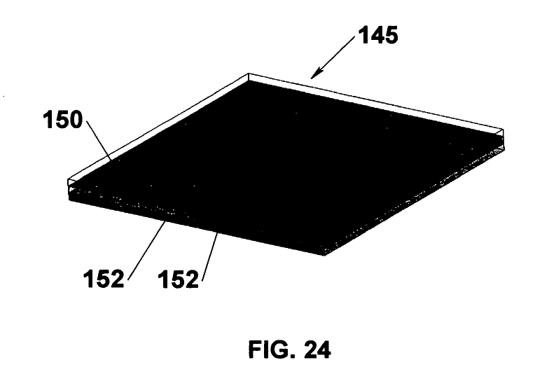

Examples of possible constructions of the membrane for a device according to the invention are illustrated in FIGS. 23 and 24. In FIG. 23, membrane 130 comprises a first layer 132 and a second layer 136 of suitable material such as, for example, polyurethane, or PVC. Disposed between first layer 130 and second layer 136 is middle layer 134 of any suitable material such as, for example, PET, nylon, Kevlar, polyimide, metal, or other suitable material. Middle layer 134 may be a solid core, but membrane layer 134 is a braided fiber structure. Accordingly, wound or woven fibers 138 confer stability, strength and wear properties, and controlled compliance.

Membrane 145 of FIG. 24 similarly comprises a first layer 150 and a second layer 152 of suitable materials. Middle layer 153 comprises a solid structure. Examples of suitable materials used in the construction of membrane 45 are set forth above in relation to FIG. 23.

While all of the foregoing embodiments can most advantageously be delivered in a minimally invasive, percutaneous manner, the foregoing embodiments may also be implanted surgically. Further, while particular forms of the invention have been illustrated and described above, the foregoing descriptions are intended as examples, and to one skilled in the art it will be apparent that various modifications can be made without departing from the spirit and scope of the invention.

1. An endoprosthesis for partial or complete replacement of an intervertebral disc comprising one or more shape memory polymers, wherein said one or more shape memory polymers is synthesized from a first monomer and a second monomer, said first and second monomers selected to impart predetermined properties on said shape memory polymer.

2. The endoprosthesis according to claim 2 wherein said first monomer and said second monomer are combined in a ratio to impart predetermined properties on said shape memory polymer.

3. The endoprosthesis according to claim 2 wherein said first monomer comprises a first molecular weight wherein said first molecular weight is a first parameter in determining said predetermined properties of said shape memory polymer.

4. The endoprosthesis according to claim 2 wherein said one or more shape memory polymers comprises one or more hard segments and one or more soft segments, said hard segments and soft segments formed from said first and second monomer and wherein said one or more hard segments comprises a first transition temperature, and said one or more soft segments comprises a second transition temperature.

5. The endoprosthesis according to claim 4 wherein said one or more hard segments comprises a transition temperature between 37° C. and 81° C., and said one or more soft segments comprises a transition temperature that is at least 10° C. less than the transition temperature of said hard segment.

6. The endoprosthesis according to claim 2 wherein said properties comprise one or more properties comprises load bearing capability, compressive resistance, stiffness, crystallinity, tensile strength, mechanical strength, durometer, elasticity, strain recovery rate, strain fixity rate, melting temperature, crystallization temperature, cross-linking density, extent of physical cross-linking, extent of covalent bond cross-linking, extent of formation of interpenetrating networks, and heat of fusion.

7. The endoprosthesis according to claim 1 wherein said shape memory polymer comprises one or more segments comprising polyurethanes, polyethylenes, fluoropolymers, thermoplastic elastomers, and composites thereof.

8. The endoprosthesis according to claim 1 wherein said endoprosthesis substantially replicates the functions of a naturally occurring, healthy intervertebral disc.

9. The endoprosthesis according to claim 1, said endoprosthesis further comprising a delivery configuration and a deployed configuration.

10. The endoprosthesis according to claim 9, said endoprosthesis further comprising a generally flat, elliptical structure, said generally flat, elliptical structure comprising a securing rim for engagement with one or more of a first and second vertebral body in a spine.

11. The endoprosthesis according to claim 10, wherein said first and second vertebral bodies each comprise a posterior region, and wherein said rim does not engage said first and second vertebral bodies at said posterior region.

12. The endoprosthesis according to claim 13, said generally flat, circular structure further comprising a top surface and a bottom surface, wherein one or more of said top and bottom surface comprises a convex portion.

13. The endoprosthesis according to claim 12, said endoprosthesis further comprising a generally disc-shaped structure, said generally disc-shaped structure comprising one or more securing tabs for engagement with one or more of a first and second vertebral body in a spine.

14. The endoprosthesis according to claim 1, wherein said endoprosthesis comprises an artificial disc nucleus for replacement of an intervertebral disc nucleus.

15. The artificial disc nucleus according to claim 14, wherein said disc nucleus comprises a durometer in the range of 20 to 70 Shore A.

16. The endoprosthesis according to claim 1, wherein said endoprosthesis comprises the capability of withstanding a mechanical load of between 800N and 6000N or more.

17. The endoprosthesis according to claim 1, wherein said endoprosthesis comprises the capability of withstanding two million or more cycles of fatigue testing.

18. The endoprosthesis according to claim 1, wherein said endoprosthesis comprises the capability of allowing range of motion of a spine of 10 degrees or more in all directions.

19. The endoprosthesis according to claim 1 wherein said one or more shape memory polymers is hydrophobic.

20. The endoprosthesis according to claim 1 wherein said one or more shape memory polymers is a thermoplastic elastomer.

21. The endoprosthesis according to claim 1 wherein said one or more shape memory polymers is a thermoset.

22. The endoprosthesis according to claim 1 wherein said one endoprosthesis comprises a generally flat, circular structure, and wherein said generally flat, circular structure comprises a central region, said central region comprising a void for receiving an artificial disc nucleus.

23. The endoprosthesis according to claim 22 wherein said endoprosthesis comprises a durometer in the range of between 20 and 70 Shore A.

24. The endoprosthesis according to claim 1, wherein said endoprosthesis substantially completely replaces an intervertebral disc, wherein said endoprosthesis comprises a nucleus region and an annulus region, and wherein said nucleus region comprises a first durometer and said annulus region comprises a second durometer, wherein said first durometer is lower than said second durometer.

25. The endoprosthesis according to claim 24, wherein said nucleus region is generally central within said endoprosthesis, said nucleus region comprises a first durometer, and wherein said prosthesis comprises a range of gradually increasing durometers, wherein said first durometer is a lowest durometer, and said gradually increasing durometers increase incrementally from said nucleus region annularly, outward throughout said annulus region.

26. The endoprosthesis according to claim 25, wherein said endoprosthesis comprises a nucleus portion and an annular portion, wherein said nucleus portion and said annulus portion are combined to form an intervertebral disc assembly.

27. The endoprosthesis according to claim 26, wherein said nucleus portion comprises a first durometer and said annulus portion comprises a second durometer, wherein said first durometer is lower than said second durometer.

28. An artificial intervertebral disc for the complete or partial replacement of an intervertebral disc comprising a delivery configuration and a deployed configuration, wherein said deployed configuration comprises a generally disc-shaped structure and wherein said artificial intervertebral disc substantially replicates the functions of a naturally occurring, healthy intervertebral disc.

29. The endoprosthesis according to claim 28 wherein said endoprosthesis comprises a durometer in the range of between 20 and 70 Shore A.

30. The endoprosthesis according to claim 28, wherein said endoprosthesis substantially completely replaces an intervertebral disc, wherein said endoprosthesis comprises a nucleus region and an annulus region, and wherein said nucleus region comprises a first durometer and said annulus region comprises a second durometer, wherein said first durometer is lower than said second durometer.

31. The endoprosthesis according to claim 30, wherein said nucleus region is generally central within said endoprosthesis, said nucleus region comprises a first durometer, and wherein said prosthesis comprises a range of gradually increasing durometers, wherein said first durometer is a lowest durometer, and said gradually increasing durometers increase incrementally from said nucleus region annularly, outward throughout said annulus region.

32. The endoprosthesis according to claim 28, wherein said endoprosthesis comprises a nucleus portion and an annular portion, wherein said nucleus portion and said annulus portion are combined to form an intervertebral disc assembly.

33. The endoprosthesis according to claim 32, wherein said nucleus portion comprises a first durometer and said annulus portion comprises a second durometer, wherein said first durometer is lower than said second durometer.

34. The artificial intervertebral disc according to claim 28, said generally flat, circular structure comprising a securing rim for engagement with one or more of a first and second vertebral body in a spine.

35. The artificial intervertebral disc according to claim 34, wherein said first and second vertebral bodies each comprise posterior portion, and wherein said securing rim does not engage said first and second vertebral bodies at said posterior portion.

36. The artificial intervertebral disc according to claim 28, said generally flat, circular structure further comprising a top surface and a bottom surface, wherein one or more of said top and bottom surface comprises a convex portion.

37. The artificial intervertebral disc according to claim 36, said generally disc-shaped structure comprising one or more securing tabs for engagement with one or more of a first and second vertebral body in a spine.

38. The artificial intervertebral disc according to claim 36, wherein said artificial intervertebral disc comprises the capability of withstanding a mechanical load of between 800N and 6000N or more.

39. The artificial intervertebral disc according to claim 36, wherein said artificial intervertebral disc comprises the capability of withstanding two million or more cycles of fatigue testing.

40. A method of manufacturing an endoprosthesis for partial or total replacement of an intervertebral disc comprising:
selecting a first monomer comprising a first set of characteristics that serves as a first parameter in determining the properties of a shape memory polymer;
selecting a second monomer comprising a second set of characteristics that serves as a second parameter in determining the properties of a shape memory polymer;
determining a desired ratio of said first monomer to said second monomer;
synthesizing a shape memory polymer from said first and said second monomer;
manufacturing an endoprosthesis for partial or total replacement of an intervertebral disc from said shape memory polymer;
setting a permanent shape for said endoprosthesis;
setting a temporary shape for said endoprosthesis.

41. The method according to claim 40 wherein said first and second sets of characteristics comprise molecular weight, transition temperature, readiness to form physical crosslinks, readiness to form covalent bonds, or crystallinity.

42. The method according to claim 40 wherein said properties of a shape memory polymer comprise extent of physical crosslinking, extent of covalent bonds, extent of networking, tensile strength, transition temperature, melting temperature, strain recovery rate, strain fixity rate, modulus of elasticity, degree of crystallization, or hydrophobicity.

43. The method according to claim 40 with the added step of:
curing said endoprosthesis according to a desired pattern.

44. The method according to claim 42 with the added step of:
increasing the degree of crystallization of said polymer according to a desired pattern.

45. The method according to claim 40 with the added step of:
cross-linking said endoprosthesis according to a desired pattern.

46. The method according to claim 40, wherein the step of setting a temporary shape includes folding the endoprosthesis into a temporary shape and constraining said endoprosthesis in said temporary shape.

47. A method of completely or partially replacing an intervertebral disc, said method comprising the steps of:
removing all or a portion of the native disc;
providing an endoprosthesis comprising one or more shape memory polymers synthesized from a first monomer and a second monomer, said first and second monomers selected to impart predetermined properties on said shape memory polymer;
delivering said endoprosthesis;
deploying said endoprosthesis.

48. The method according to claim 47, wherein the step of removing all or a portion of the native disc does not include removing the periphery of the native annulus fibrosus.

49. The method according to claim 47, wherein the step of removing all or a portion of the native disc includes removal of the native nucleus only, and wherein the step of delivering an endoprosthesis comprises delivering an artificial nucleus pulposus.

50. The method according to claim 47, wherein said step of delivering an endoprosthesis comprises delivering an artificial annulus fibrousus, followed by the delivery of an artificial nucleus pulposus.

51. The method according to claim 47, wherein said step of removing all or a portion of said native intervertebral disc comprises removing substantially all of said native intervertebral disc, and said step of percutaneously delivering said endoprosthesis comprises delivering a complete replacement artificial disc.

52. The method according to claim 47, wherein said method is performed surgically.

53. The method according to claim 52, wherein said method is performed surgically from an anterior approach.

54. The method according to claim 47, wherein said method is performed percutaneously.

55. The method according to claim 54, wherein said method is performed percutaneously from a posterior approach.

56. The method according to claim 47, wherein said endoprosthesis comprises one or more constraints, and said step of deploying said endoprosthesis comprises removing said one or more constraints.

57. The method according to claim 47, wherein said step of deploying said endoprosthesis comprises exposing said endoprosthesis to one or more initiators.

58. A method of completely or partially replacing an intervertebral disc, said method comprising the steps of:
removing all or a portion of the native disc;
providing an endoprosthesis comprising one or more superelastic polymers synthesized from a first monomer and a second monomer, said first and second monomers selected to impart predetermined properties on said superelastic polymer;
percutaneously delivering said endoprosthesis;
deploying said endoprosthesis.

59. The method according to claim 58, wherein the step of removing all or a portion of the native disc does not include removing the periphery of the native annulus fibrosus.

60. The method according to claim 58, wherein the step of removing all or a portion of the native disc includes removal of the native nucleus only, and wherein the step of delivering an endoprosthesis comprises delivering an artificial nucleus pulposus.

61. The method according to claim 58, wherein said step of delivering an endoprosthesis comprises delivering an artificial annulus fibrousus, followed by the delivery of an artificial nucleus pulposus.

62. The method according to claim 58, wherein said step of removing all or a portion of said native intervertebral disc comprises removing substantially all of said native intervertebral disc, and said step of percutaneously delivering said endoprosthesis comprises delivering a complete replacement artificial disc.

63. The method according to claim 58, wherein said method is performed surgically.

64. The method according to claim 63, wherein said method is performed surgically from an anterior approach.

65. The method according to claim 58, wherein said method is performed percutaneously.

66. The method according to claim 65, wherein said method is performed percutaneously from a posterior approach.

67. The method according to claim 58, wherein said endoprosthesis comprises one or more constraints, and said step of deploying said endoprosthesis comprises removing said one or more constraints.

68. An artificial disc comprising one or more substantially hollow bodies, a delivery configuration and a deployed configuration, wherein said one or more substantially hollow bodies is placed in said deployed configuration upon the introduction of a material into said one or more substantially hollow bodies.

69. The artificial disc according to claim 68 wherein said artificial disc is placed in its deployed configuration after it is delivered to a treatment site.

70. The artificial disc according to claim 68 wherein said artificial disc comprises an artificial annulus component and an artificial nucleus component.

71. The artificial disc according to claim 68 wherein said one or more substantially hollow bodies comprises a membrane comprising one or more layers.

72. The artificial disc according to claim 71 wherein said one or more layers comprises one or more material from the group consisting of polyurethane, polyethylene terephthalate, polyvinyl chloride, nylon, Kevlar, polyimide, and metal.

73. The artificial disc according to claim 68 wherein said artificial disc comprises a filling material when in its deployed configuration.

74. The artificial disc according to claim 73 wherein said filling material comprises one or more materials from the group consisting of saline, contrast medium, hydrogel, perfluoropolyethers and polymeric foam.

75. The artificial disc according to claim 74 wherein said polymeric foam comprises a polymeric diisocyanate, polyol and hydrocarbon.

76. The artificial disc according to claim 74 wherein said polymeric foam comprises carbon dioxide.

77. The artificial disc according to claim 72 wherein one or more layers comprises a braided fiber structure.

78. The artificial disc according to claim 77 wherein said braided fiber structure is disposed between two or more solid layers.

79. The artificial disc according to claim 68 further comprising one or more injection ports.

80. The artificial disc according to claim 70 wherein said artificial nucleus comprises an injection port and said artificial annulus comprises an injection port.

81. The artificial disc according to claim 80 wherein said artificial disc, when in its deployed configuration, comprises a first filling medium within said artificial nucleus, and a second filling medium within said artificial annulus.

82. The artificial disc according to claim 81 wherein said first filling medium confers on said artificial nucleus properties similar to a native nucleus pulposus, and said second filling medium confers properties on said artificial annulus similar to a native annulus fibrosus.

83. An artificial nucleus comprising one or more substantially hollow bodies, a delivery configuration and a deployed configuration, wherein said one or more substantially hollow bodies is placed in said deployed configuration upon the introduction of a material into said one or more substantially hollow bodies.

84. An artificial annulus comprising one or more substantially hollow bodies, a delivery configuration and a deployed configuration, wherein said one or more substantially hollow bodies is placed in said deployed configuration upon the introduction of a material within said one or more substantially hollow bodies.

85. The artificial disc according to claim 75 wherein said polymeric foam comprises one or more additional gases.

86. The artificial disc according to claim 68 wherein said one or more of said substantially hollow bodies comprises one or more means for directing flow of said material within said substantially hollow bodies.

87. The artificial disc according to claim 86 wherein one or more of said means for directing flow of said material comprises one or more inverted seams.

88. The artificial disc according to claim 68 wherein said one or more of said substantially hollow bodies comprises one or more interbody connections.

89. An artificial disc nucleus comprising one or more hollow bodies, one or more chambers within said one or more hollow bodies, and one or more materials within the interior of one or more of said hollow bodies, wherein said artificial disc nucleus further comprises one or more materials formed from a polymer synthesized from a first monomer and a second monomer to impart shape memory characteristics upon said material.

90. An artificial disc or disc nucleus for the treatment of a degenerated or traumatized intervertebral disc, said disc or nucleus comprising a durometer selected for the level within the spine of the disc undergoing treatment.
